(19) United States
(12) Patent Application Publication
(10) Pub. No.: US 2003/0118652 A1
(43) Pub. Date: Jun. 26, 2003

(54) Methods and compositions for use of (S)-bisoprolol

(76) Inventors: John Kelly, Drumcondra (IE); John Devane, Athlone (IE)

Correspondence Address: FINNEGAN, HENDERSON, FARABOW, GARRETT & DUNNER, LLP, 1300 I STREET, NW, WASHINGTON, DC, 20005, US

(21) Appl. No.: 10/294,693

(22) Filed: Nov. 15, 2002

Related U.S. Application Data
(63) Continuation-in-part of application No. 10/225,543, filed on Aug. 22, 2002.
(60) Provisional application No. 60/335,884, filed on Nov. 15, 2001.

(51) Int. Cl. A61K 31/137; A61K 9/24

(52) U.S. Cl. 424/473; 514/651

(57) ABSTRACT

Methods of treating, preventing, and/or managing cardiovascular conditions such as hypertension, ischemic heart disease, atrial fibrillation, congestive heart failure, angina pectoris, and cardiac arrhythmias, with enriched (S) stereoisomer of bisoprolol are disclosed, as are compositions and formulations comprising enriched (S)-bisoprolol.

This application is a continuation-in-part of U.S. application Ser. No. 10/225,543, filed Aug. 22, 2002, and further claims priority to U.S. Provisional Patent Application No. 60/335,884, filed Nov. 15, 2001. The entire disclosure of each of the aforementioned applications is incorporated herein by reference.

Bisoprolol (1-[4-[[2-(1-methylethoxy)ethoxy]-methyl]phenoxy]-3-[(1-methylethyl)amino]-2-propanol), in its racemic form, is a commercially available drug that acts as a β-adrenergic blocking agent (antagonist). The drug acts by blocking neurotransmitter action at β-adrenergic receptors and, as a consequence, disrupts sympathetic nervous system transmission. The effects of β-adrenergic blockade are widespread, reflecting the distribution of these receptors throughout the body. They include, but are not limited to, effects on the heart and cardiovascular system, the gastrointestinal tract, the respiratory tract, the eye, the liver, and the genitourinary system. These effects and others are described, for example, in textbooks such as Goodman and Gilman's The Pharmacological Basis of Therapeutics (McGraw Hill, 1996) and Rang, Dale and Ritter's Pharmacology (Churchill Livingstone, 1999).

The β-adrenergic antagonists are indicated for a number of conditions including, but not limited to, hypertension, ischemic heart disease, atrial fibrillation, congestive heart failure, peripheral arterial occlusive disease, angina pectoris, cardiac dysrhythmias, heart failure, glaucoma, migraine, the effects of thyroid disease, and symptoms of anxiety, such as palpitations. The antagonists are most commonly used in treatment of diseases of the cardiovascular system.

Despite the clinical usefulness of β-adrenergic antagonists, including bisoprolol, there are well-documented side effects that limit their dose or restrict their use. Some side effects, such as slow or irregular heart rate or fatigue, result from the primary effects of these drugs. Others, such as bronchial constriction, impaired circulation to the extremities, hypoglycemia, and blunting of hyperglycemic awareness in diabetes, involve secondary effects caused by the drug's blockade of other subtypes of the β-adrenergic receptors, such as the β-2adrenergic receptors. Still other side effects, such as rashes, are not believed to be specific to any particular action of the drug on the β-adrenergic receptor system.

For example, racemic bisoprolol has been reported to cause bronchoconstrictor effects, including wheeziness and breathlessness (Dorow et al., Eur. J. Clin. Pharmacol. 31(2):143-7: 1986), adverse side effects on the peripheral circulation (Bailliart et al., Eur Heart J; 8 Suppl M:87-93: 1987), and effects on carbohydrate metabolism that are deleterious to patients with diabetes (Janka et al., J. Cardiovasc. Pharmacol; 8 Suppl 11:S96-9: 1986).

The main clinical indications of hypertension, angina pectoris, and heart failure, are mediated by β-1 receptors. However, as noted above, some of the undesired effects, such as bronchoconstriction, peripheral vasoconstriction, and hypoglycemia, are mediated largely by β-2 receptors.

Racemic bisoprolol exhibits a selectivity for the β-1 adrenergic receptor (over the β-2 and/or β-3 adrenergic receptors) that is two to thirty times higher than the selectivity exhibited by other commonly used β-blockers, such as atenolol and propranolol (Schliep et al., Eur. J. Pharmacol ., 123(2):253-61, 1986; Wellstein et al., Eur. Heart J., 8(Suppl. M):3-8, 1987; Schnabel et al., J. Cardiovasc. Pharmacol., 36(4):466-71, 2000; Klockow et al., Arzneimittelforschung, 36(2):197-200, 1986). For, example, racemic bisoprolol's selectivity for the β-1 adrenergic receptor is at least double that of atenolol, which is commonly recognized for its selectivity against the β-1 receptor (Klockow et al., Id.; Wellstein et al., Id.). At higher doses, however, the selectivity of racemic bisoprolol for the β-1 adrenergic receptor is reportedly reduced (Haffner et al., J. Hum. Hypertens., 6(5):397-400, 1992).

Despite this general selectivity, some patients being treated with racemic bisoprolol experience unwanted side effects at the doses needed for therapeutic efficacy. Thus, there exists a need in the art for safer and more effective β-adrenergic blocking agents and/or formulations that achieve the desirable clinical benefits, while minimizing the undesirable side effects.

Many drug compounds contain chiral centers and exist in at least two different stereoisomeric forms. Some of these stereoisomers are related as non-superimposable, mirror images known as enantiomers. Individual enantiomers are also known as racemates. A 1:1 mixture of racemates is also known as a racemic mixture. A mixture of enantiomers in which one is present in an amount greater than the other is called an enantiomerically enriched (hereafter “enriched”) mixture. Generally, compounds containing chiral centers, which are used as drugs, are provided in the form of a 1:1 mixture of enantiomers, in other words, a racemic mixture. For example, bisoprolol is normally provided as a racemic mixture, containing approximately equal amounts of its two enantiomers, (R)-bisoprolol and (S)-bisoprolol.

Molecular modeling studies suggest that the (S)-enantiomer of bisoprolol, like other β-1 adrenergic receptor blockers, assumes a conformation in which its aromatic, amino, and β-hydroxyl groups are believed to interact with certain amino acid side chains in the β-1 adrenergic receptor (Nandel et al., Ind. J. Biochem. Biophys., 32(4); 207-212, 1995 and Nandel et al., Ind. J. Biochem. Biophys., 36(1):29-35, 1999). Following administration of racemic bisoprolol, the area under the plasma concentration-time curve and the elimination half-life are slightly higher for (S)-bisoprolol than for (R)-bisoprolol (Horikiri et al., J. Pharm. Sci., 87(3): 289-294, 1998).

The present invention is directed to new compositions that comprise enriched (S)-bisoprolol and methods of their use. Although not wishing to be bound by any particular theory, it is believed that the presence of (R)-bisoprolol in racemic bisoprolol reduces the overall selectivity of the drug because it interacts non-specifically with both the β-1 and β-2 receptor subtypes. This non-specific interaction is believed to be the cause of undesirable side effects associated with the administration of racemic bisoprolol. It is believed that (S)bisoprolol is relatively selective for the β-1 receptor. Thus, the administration of enriched (S)-bisoprolol may cause fewer side-effects in patients receiving it than those who receive the racemic mixture. Thus, the enriched (S)-bisoprolol compositions of the present invention provide several important advantages compared to racemic bisoprolol compositions as well as other β-adrenergic antagonists.

For example, the total amount of drug product needed to achieve a desired therapeutic effect may be lower when enriched (S)-bisoprolol is used, relative to the racemic mixture. For example, the amount of enriched (S)-bisoprolol may be less than 90, 80, 70, or less than 50% of the amount of racemic bisoprolol needed to achieve the same effect. Thus, a lower amount of total drug product can be used in the final formulations. Lower amounts of total drug product can minimize a patient's exposure to xenobiotic substances, thereby reducing many side effects and providing increased safety. There can also be a lesser potential for non-specific side effects, such as skin rashes. In addition, the final formulation, such as a tablet, may be made smaller and thus easier to swallow.

Another advantage of using enriched (S)-bisoprolol as compared to an equivalent weight of the racemic mixture is a prolonged therapeutic effect. It is believed that (R)-bisoprolol is oxidatively metabolized by the cytochrome P450 isoform CYP2D6 at a greater rate than is (S)-bisoprolol. In addition, the rate of renal clearance is greater for (R)-bisoprolol than it is for (S)-bisoprolol. Therefore, a prolonged therapeutic effect is expected for those patients receiving a composition comprising enriched (S)-bisoprolol as compared to those receiving the same dose of racemic bisoprolol.

Additionally, the present invention may reduce adverse drug interactions. As noted above, it is believed that (R)-bisoprolol is more extensively metabolized by the CYP2D6 isoform of P450. Consequently, drugs that compete for this same enzyme isoform in order to be metabolized and cleared from a patient's body may accumulate in the blood and increase the likelihood of adverse drug interactions.

In addition, polymorphic differences in the expression of the CYP2D6 isoform can result in significant variability in clearance, in particular, of the (R)-isomer, resulting in significant inter-patient variation in systemic exposure. Along the same lines, there are drugs that are known to induce or inhibit the activity of the CYP2D6 isoform, which when co-administered with racemic verapamil, result in additional variability of the systemic exposure.

The enriched (S)-bisoprolol compositions according to the present invention may also be prepared as more safe and effective dosage forms, such as once-daily, controlled-release dosage forms that exhibit lower peak-to-trough fluctuations in the plasma concentrations of the compound. This allows for the avoidance of pronounced peak concentrations, keeping plasma concentration within ranges that are optimal for (S)-bisoprolol's β-1 adrenergic receptor selectivity. By maintaining this optimal range, the potential for side effects due to β-2 receptor antagonism is reduced.

Suitable dosage forms may be prepared in any manner, and may be prepared as delayed-release formulations, including chronotherapeutic formulations described in detail below. Such formulations are designed to relieve the early morning pathologies reported by patients suffering from various cardiovascular conditions. It is well documented that there is an increased risk of sudden death, myocardial infarction, and acute cerebrovascular events in the morning hours for such patients. Additionally, these patients often experience discomfort just before, and for the first few hours after, awakening. To avoid or relieve these symptoms, the present enriched (S)-bisoprolol formulations can be designed to provide a patient with therapeutically effective levels of bisoprolol just prior to awakening, during the early morning hours, and throughout the day.

Given these unique features of the enriched (S)-bisoprolol compositions according to the present invention, the compositions provide significant advantages in the treatment, prevention, and/or management of cardiovascular conditions such as hypertension, angina, and/or heart failure. The compositions also minimize unwanted side effects associated with the use of racemic bisoprolol.

Accordingly, the present invention relates to new compositions and methods for preventing, treating, and/or managing one or more cardiovascular conditions using a stereo-specific form of bisoprolol. The compositions and methods are based on the discovery that enriched (S)-bisoprolol exhibits a greater effect on components of the cardiovascular system than does (R)-bisoprolol. In addition, enriched (S)-bisoprolol exhibits greater selectivity for the β-1 adrenergic receptor than does the racemic mixture. Accordingly, enriched (S)-bisoprolol formulations provide the advantages discussed above as well as a greater safety and efficacy profile than the racemic mixture.

As used herein, the term “pharmaceutically acceptable excipient” includes compounds that are compatible with the other ingredients in a pharmaceutical formulation and not injurious to the subject when administered in therapeutically effective amounts.

As used herein, the term “pharmaceutically acceptable salt” includes salts that are physiologically tolerated by a subject. Such salts are typically prepared from an inorganic or organic acid. Examples of suitable inorganic acids include, but are not limited to, hydrochloric, hydrobromic, hydroiodic, nitric, sulfuric, and phosphoric acid. Organic acids may be aliphatic, aromatic, carboxylic, and/or sulfonic acids. Suitable organic acids include, but are not limited to, formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic, and the like.

The term “racemic”, as used herein, means a mixture of (R)-and (S)-bisoprolol in which neither isomer is substantially purified from the other.

The term “enriched (S)-bisoprolol”, as used herein, refers to a composition in which (S)-bisoprolol is present in an amount greater than (R)-bisoprolol.

In one embodiment, the enriched (S)-bisoprolol compositions comprise (S)-bisoprolol in an amount greater than (R)-bisoprolol. For example, the enriched (S)-bisoprolol compositions may comprise (S)-bisoprolol in the range of greater than about 50% by weight, relative to the total weight of bisoprolol.

In another embodiment, the enriched (S)-bisoprolol compositions comprise (S)-bisoprolol in the range of about 50-60% by weight, relative to the total weight of bisoprolol.

In another embodiment, (S)-bisoprolol may be, for example, at least about 52%, 53%, 54%, 55%, 60%, 65%, 70%, 75%, 80%, 81%, 82%, 83%, 84%, 85%. 86%, 87%, 88%, 89%, 90%, 91%, 92%, 93%, 94%, 95%, 96%, 97%, 98%, 99%, or 100%, or any fraction thereof (i.e., 90.1%, 90.2%, etc.), by weight, relative to the total weight of bisoprolol. These terms and ranges also apply to the amount of pharmaceutically acceptable salts of bisoprolol.

The phrase “therapeutically effective amount” of enriched (S)-bisoprolol, as used herein, refers to the amount of enriched (S)-bisoprolol (or pharmaceutically acceptable salt thereof), which alone or in combination with other drugs, provides any therapeutic benefit in the prevention, treatment, and/or management of one or more conditions described below, which are amenable to prevention, treatment, and/or management with racemic bisoprolol. In one embodiment, the therapeutic amount is sufficient to achieve a therapeutic benefit for these conditions while reducing or avoiding one or more of the unwanted side effects listed above that are typically associated with administration of racemic bisoprolol. In some embodiments, the therapeutic amount of enriched (S)-bisoprolol used in the treatment, prevention, and/or management of one or more of the above-specified conditions is equal to or lower than the therapeutic amount required when using the racemic form of the drug to prevent, treat, and/or manage the same condition.

The present invention is directed to compositions and methods for preventing, treating, and/or managing cardiovascular conditions that are preventable, treatable, and/or manageable with racemic bisoprolol. The methods involve administering a therapeutically effective amount of enriched (S)-bisoprolol, or a pharmaceutically acceptable salt thereof, to a subject in need of such prevention, treatment, and/or management. The cardiovascular conditions that may be prevented, treated, and/or managed using the inventive compositions and methods include, but are not limited to, hypertension, ischemic heart disease, atrial fibrillation, cardiac arrhythmias, congestive heart failure, angina pectoris, migraine headaches, and cardiovascular manifestations of anxiety, such as palpitations. The compositions and methods may also be used as an adjunctive therapy for hyperthyroidism. Other conditions involving abnormal cardiovascular activity may also be treated, prevented, or managed using the presently disclosed compositions and methods.

Enriched (S)-bisoprolol may be obtained from a racemic mixture of bisoprolol (see, e.g., Kim, et al., Arch. Pharm. Res., 24(5):402-6, 2001, which is incorporated herein by reference). It may be obtained from racemic mixtures by high performance liquid chromatography (HPLC) separation, or resolution of the enantiomers using any means, such as an optically active resolving acid. In addition, enriched (S)-bisoprolol may be synthesized using any appropriate stereoselective methodology, examples of which are well-known to those of ordinary skill in the art (see, e.g., Kitaori, et al., Tetrahedron Lett., 39(20), 3173-3176, 1998, which is incorporated herein by reference). Stereoselective synthesis methods may be combined with additional separation techniques to further increase the enantiomeric purity of enriched (S)-bisoprolol compositions. Examples of processes for resolving racemic bisoprolol to obtain enriched (S)-bisoprolol are well known to those of ordinary skill in the art.

The invention also includes compositions and methods of their use in preventing, treating, and/or managing one or more cardiovascular conditions. Accordingly, the invention includes a method of preventing, treating, and/or managing at least one cardiovascular condition by administering a therapeutically effective amount of enriched (S)-bisoprolol, or a pharmaceutically acceptable salt thereof, to a subject in need of such a treatment, prevention, and/or management. In one embodiment, the administration of enriched (S)-bisoprolol or pharmaceutically acceptable salt thereof reduces one or more side effects relative to those observed following administration of a racemic mixture of bisoprolol.

In another embodiment, the present invention relates to methods of reducing side effects associated with the administration of racemic bisoprolol comprising administering a therapeutically effective amount of enriched (S)-bisoprolol, or a pharmaceutically acceptable salt thereof, to a subject in need of such prevention, treatment, and/or management, wherein one or more side effects are reduced relative to those resulting from the administration of an equivalent amount of the racemic bisoprolol.

The invention also includes compositions and methods of use of enriched (S)-bisoprolol to achieve the same therapeutic effect relative to the amount required when the racemic mixture is used. Accordingly, the invention includes methods of preventing, treating, and/or managing one or more cardiovascular conditions comprising administering a therapeutically effective amount of enriched (S)-bisoprolol, or a pharmaceutically acceptable salt thereof, to a subject in need of prevention, treatment, and/or management, wherein the subject obtains a therapeutic benefit resulting from the administration of enriched (S)-bisoprolol, and wherein the amount of enriched (S)-bisoprolol, or pharmaceutically acceptable salt thereof, is less than the amount required to achieve the same therapeutic benefit using a racemic mixture of bisoprolol.

The invention also includes compositions, and methods of their use, which reduce drug interactions in subjects receiving the formulations. Accordingly, the present invention includes methods of reducing drug interactions associated with racemic bisoprolol, comprising administering a therapeutically effective amount of enriched (S)-bisoprolol, or a pharmaceutically acceptable salt thereof, to a subject in need of such a treatment, prevention and/or management wherein one or more drug interactions are reduced relative to those resulting from the administration of an equivalent amount of racemic bisoprolol.

In yet another embodiment, the present invention includes compositions, and methods of their use, which reduce the oxidative metabolism associated with the cytochrome P450 isoform CYP2D6. Accordingly, the present invention includes a method of administering a therapeutically effective amount of enriched (S)-bisoprolol, or a pharmaceutically acceptable salt thereof, to a subject in need of such a prevention, treatment, and/or management, wherein the amount of oxidative metabolism by the cytochrome P450 isoform CYP2D6 in the subject is reduced relative to the amount of oxidative metabolism by CYP2D6 in a subject receiving an equal dose of racemic bisoprolol.

The invention also includes compositions, and methods of their use, which extend the therapeutic effect of a treatment for one or more cardiovascular conditions. Accordingly, the invention includes a method of extending the therapeutic effect of a bisoprolol treatment comprising administering a therapeutically effective amount of enriched (S)-bisoprolol, or a pharmaceutically acceptable salt thereof, to a subject in need of such a treatment, wherein the administration of enriched (S)-bisoprolol, or pharmaceutically acceptable salt thereof, provides a therapeutic effect that lasts longer than that achieved after administration of an equal amount of racemic bisoprolol.

The enriched (S)-bisoprolol, or pharmaceutically acceptable salt thereof, may be provided in a pharmaceutical composition for use according to the present invention. Such compositions optionally include one or more pharmaceutically acceptable excipients. Suitable excipients are known to those of skill in the art and are described, for example, in the Handbook of Pharmaceutical Excipients (Kibbe (ed.), 3^{rd} Edition (2000), American Pharmaceutical Association, Washington, D.C.), and Remington's Pharmaceutical Sciences (Gennaro (ed.), 20^{th} edition (2000), Mack Publishing, Inc., Easton, Pa.), which, for their disclosures relating to excipients and dosage forms, are incorporated herein by reference.

Suitable excipients include, but are not limited to, starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, plasticizers, gelling agents, thickeners, hardeners, setting agents, suspending agents, surfactants, humectants, carriers, stabilizers, antioxidants, and combinations thereof.

The pharmaceutical compositions of the invention are typically provided in dosage forms that are suitable for administration to a subject by a desired route. A number of suitable dosage forms are described below, but this description is not meant to include all possible choices. One of skill in the art is familiar with the various dosage forms that are suitable for use in the present invention, as described, for example, in Remington's Pharmaceutical Sciences, portions of which have been incorporated by reference above. The most suitable route in any given case will depend on the nature and severity of the condition being prevented, treated, and/or managed. The pharmaceutical compositions of this invention may be formulated for administration orally, nasally, rectally, intravaginally, parenterally, intracisternally, and topically (including buccally and sublingually).

Formulations suitable for oral administration include, but are not limited to, capsules, cachets, pills, tablets, lozenges (using a flavored base, usually sucrose and acacia or tragacanth), powders, granules, solutions, suspensions in an aqueous or non-aqueous liquid, oil-in-water or water-in-oil liquid emulsions, elixirs, syrups, pastilles (using an inert base, such as gelatin and glycerin, or sucrose and acacia), mouth washes, pastes, and the like, each containing a predetermined amount of enriched (S)-bisoprolol to provide a therapeutic amount of the drug in one or more doses.

Enriched (S)-bisoprolol is typically mixed with one or more pharmaceutically-acceptable excipients in the preparation of solid dosage forms for oral administration (capsules, tablets, pills, powders, granules and the like). Suitable excipients include, but are not limited to, carriers, such as sodium citrate or dicalcium phosphate; fillers or extenders, such as starches, lactose, sucrose, glucose, mannitol, or silicic acid; binders, such as hydroxymethyl-cellulose, alginates, gelatin, polyvinylpyrrolidone, sucrose or acacia; humectants, such as glycerol; disintegrating agents, such as agar, calcium carbonate, potato or tapioca starch, alginic acid, certain silicates, or sodium carbonate; solution retarding agents, such as paraffin; absorption accelerators, such as quaternary ammonium compounds; wetting agents, such as cetyl alcohol or glycerol monostearate; absorbents, such as kaolin and bentonite clay; lubricants, such as talc, calcium stearate, magnesium stearate, solid polyethylene glycols, and sodium lauryl sulfate; coloring agents; buffering agents; dispersing agents; preservatives; and diluents. The aforementioned excipients are given as examples only and are not meant to include all possible choices. Solid compositions may also be employed as fillers in soft and hard-filled gelatin capsules using excipients such as lactose or milk sugars, high molecular weight polyethylene glycols, and the like. Any of these dosage forms may optionally be scored or prepared with coatings and shells, such as enteric coatings and coatings for modifying the rate of release, examples of which are well known in the pharmaceutical-formulating art.

For oral administration, enriched (S)-bisoprolol may be formulated into a liquid dosage form. Suitable formulations include emulsions, microemulsions, solutions, suspensions, syrups, and elixirs. These formulations optionally include diluents commonly used in the art, such as, for example, water or other solvents, solubilizing agents and emulsifiers, including, but not limited to, ethyl alcohol, isopropyl alcohol, ethyl carbonate, ethyl acetate, benzyl alcohol, benzyl benzoate, propylene glycol, 1,3-butylene glycol, oils, glycerol, tetrahydrofurfuryl alcohol, polyethylene glycols, fatty acid esters of sorbitan, and mixtures thereof. In addition, the liquid formulations optionally include adjuvants such as wetting agents, emulsifying and suspending agents, sweetening, flavoring, coloring, perfuming, and preservative agents. Suitable suspension agents include, but are not limited to, ethoxylated isostearyl alcohols, polyoxyethylene sorbitol and sorbitan esters, microcrystalline cellulose, aluminum metahydroxide, bentonite, agar-agar and tragacanth, and mixtures thereof. The liquid formulations may be delivered as-is, or may be provided in hard or soft capsules, for example.

For rectal or vaginal administration, the composition may be provided as a suppository. Suppositories optionally include one or more non-irritating excipients, for example, polyethylene glycol, a suppository wax, or a salicylate. Such excipients may be selected based on desirable physical properties. For example, a compound that is solid at room temperature but liquid at body temperature will melt in the rectum or vaginal cavity and release the active compound. The formulation may alternatively be provided as an enema for rectal delivery. Formulations suitable for vaginal administration also include pessaries, tampons, creams, gels, pastes, foams, or spray formulations containing such carriers, examples of which are known in the art.

Formulations suitable for topical or transdermal administration include powders, sprays, ointments, pastes, creams, lotions, gels, solutions, patches, and inhalants. Such formulations optionally contain excipients such as animal and vegetable fats, oils, waxes, paraffins, starch, tragacanth, cellulose derivatives, polyethylene glycols, silicones, bentonites, silicic acid, talc, zinc oxide, or mixtures thereof. Powders and sprays may also contain excipients such as lactose, talc, silicic acid, aluminum hydroxide, calcium silicates, and polyamide powder. Additionally, sprays may contain propellants, such as chlorofluoro-hydrocarbons and volatile unsubstituted hydrocarbons, such as butane and propane.

Transdermal patches have the added advantage of providing controlled delivery of the mixture of the invention to the body. Such dosage forms can be made by dissolving, dispersing, or otherwise incorporating a pharmaceutical composition containing enriched (S)-bisoprolol in a suitable medium, such as an elastomeric matrix material. Absorption enhancers can also be used to increase the flux of the mixture across the skin. The rate of such flux may be controlled by providing a rate-controlling membrane or dispersing the compound in a polymer matrix or gel.

For parenteral administration, such as administration by injection (including, but not limited to, subcutaneous, bolus injection, intramuscular, intraperitoneal, and intravenous), the pharmaceutical compositions may be formulated as isotonic suspensions, solutions, or emulsions, in oily or aqueous vehicles, and may contain formulatory agents such as suspending, stabilizing, or dispersing agents. Alternatively, the compositions may be provided in dry form such as a powder, crystalline, or freeze-dried solid, for reconstitution with sterile pyrogen-free water or isotonic saline before use. They may be presented, for example, in sterile ampoules or vials.

Examples of suitable aqueous and nonaqueous excipients include water, ethanol, polyols (such as glycerol, propylene glycol, polyethylene glycol, and the like), oils, injectable organic esters, and mixtures thereof. Proper fluidity can be maintained, for example, by the use of surfactants.

These compositions may also contain adjuvants such as preservatives, wetting agents, emulsifying agents, and dispersing agents. Prevention of the action of microorganisms may be achieved by the inclusion of various antibacterial and/or antifungal agents, for example, paraben, chlorobutanol, phenol sorbic acid, and the like. It also may be desirable to include isotonic agents, such as sugars, sodium chloride, and the like in the compositions. In addition, prolonged absorption of the injectable pharmaceutical form may be brought about by the inclusion of agents that delay absorption, such as aluminum monostearate and/or gelatin.

To prolong or extend the therapeutic effect of a drug, it may be desirable to slow the absorption of the drug from a subcutaneous and intramuscular injection. This may be accomplished by the use of a liquid suspension of crystalline or amorphous material having low solubility. The rate of absorption of the drug then generally depends upon its rate of dissolution, which may depend upon crystal size and crystalline form. Alternatively, delayed absorption of a parenterally-administered form can be accomplished by dissolving or suspending the drug in an oil vehicle.

In addition to the common dosage forms discussed above, the pharmaceutical compositions may also be administered by controlled release delivery devices, examples of which are well known to those of ordinary skill in the art. Examples of different formulations are provided in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, the disclosures of which, for their discussions of pharmaceutical formulations, are incorporated herein by reference. Advantages of controlled-release formulations may include extended activity of the drug, reduced dosage frequency, and increased patient compliance. Suitable components (e.g., polymers, excipients, etc.) for use in controlled release formulations, and methods of producing the same, are also described, e.g., in U.S. Pat. No. 4,863,742, which is incorporated by reference for these purposes.

Examples of controlled or extended-release formulations include but are not limited to, diffusion-controlled, matrix, osmotic, and ionic exchange systems. These can be in the form of single (monolithic) or multiunit dosage forms. With diffusion-controlled extended release dosage forms, the formulation containing the active substance of interest, i.e., (S)-bisoprolol, may be surrounded by a semi-permeable membrane. Semi-permeable membranes include those that are permeable to a greater or lesser extent to both water and solute. This membrane may include water-insoluble and/or water-soluble polymers, and may exhibit pH-dependent and/or pH-independent solubility characteristics. Polymers of these types are described in detail below. Generally, the characteristics of the polymeric membrane (e.g., the composition of the membrane) will determine the nature of release from the dosage form.

In an osmotic-release system, a selectively permeable membrane encloses a reservoir of the substance of interest, i.e., (S)-bisoprolol, at a concentration sufficient to provide an osmotic pressure above a threshold level. Selectively permeable membranes include those that are permeable to water but not to solute. The pore or orifice size of a selectively permeable membrane can be varied so that passage of molecules of the substance through the pore or orifice of the membrane becomes the rate-limiting factor in dispensing the substance into the surrounding environment outside of the dosage form. Alternatively, the reservoir of the substance, in addition to the active ingredient, may also include an inactive substance, such as an osmotic agent, which is present at a concentration sufficient to provide an osmotic pressure above a threshold level. The active substance of interest can be present as a solid or liquid contained within the dosage form.

Matrix-type systems comprise an active substance of interest, i.e., (S)-bisoprolol, mixed with either water-soluble, e.g., hydrophilic polymers, or water-insoluble, e.g., hydrophobic polymers. Generally, the properties of the polymer used in a modified-release dosage form will affect the mechanism of release. For example, the release of the active ingredient from a dosage form containing a hydrophilic polymer can proceed via both surface diffusion and/or erosion. Mechanisms of release from pharmaceutical systems are well known to those skilled in the art. Matrix-type systems can also be monolithic or multiunit, and may be coated with water-soluble and/or water-insoluble polymeric membranes, examples which are described above.

The inventive extended release formulations may rely on ion exchange resins for the release of enriched (S)-bisoprolol. In such formulations, the drug is bound to ion exchange resins and, when ingested, the release of drug can be determined by the ionic environment within the gastrointestinal tract.

Depending on the particular need, the inventive formulations may be prepared as tablets, pellets, minitablets, caplets, or any other desired form. Any desired form may be coated or uncoated, and the coatings may exhibit a pH-dependent or pH-independent dissolution. The particular form depends upon the desired end use and the choice is left to the practitioner.

Pellet dosage forms can be, for example, encapsulated, prepared as a tablet, or administered in a food or drink. One of the advantages of encapsulated pelleted products is that the onset of absorption is less sensitive to stomach emptying. The entrance of the pellets into the small intestine can be more uniform than with non-disintegrating extended-release tablet formulations.

The release of (S)-bisoprolol can be slowed or controlled by using, for example, hydroxypropylmethyl cellulose in varying proportions to provide the desired release profile, other polymer matrices, gels, permeable membranes, osmotic systems, multilayer coatings, microparticles, liposomes, microspheres, or the like, or combinations thereof. Examples of suitable controlled-, delayed-, and/or extended-release formulations are known to those of ordinary skill in the art, and may readily be selected for use with the enriched (S)-bisoprolol compositions of the present invention. Thus, tablets, capsules, gelcaps, caplets, and the like, that are adapted for controlled-release, may be used in accordance with the presently disclosed methods. The controlled-release of the active ingredient may be triggered or stimulated by various inducers, for example pH, temperature, enzymes, water, or other physiological conditions or compounds.

The controlled release formulations used in the present methods may include any number of pharmaceutically acceptable excipients. Suitable excipients include, but are not limited to, carriers, such as sodium citrate or dicalcium phosphate; fillers or extenders, such as stearates, silicas, gypsum, starches, lactose, sucrose, glucose, mannitol, talc, or silicic acid; binders, such as hydroxymethyl-cellulose, alginates, gelatin, polyvinyl pyrrolidone, sucrose or acacia; humectants, such as glycerol; disintegrating agents, such as agar, calcium carbonate, potato or tapioca starch, alginic acid, certain silicates, or sodium carbonate; solution retarding agents, such as paraffin; absorption accelerators, such as quaternary ammonium compounds; wetting agents, such as cetyl alcohol or glycerol monostearate; absorbents, such as kaolin and bentonite clay; lubricants, such as talc, calcium stearate, magnesium stearate, solid polyethylene glycols, and sodium lauryl sulfate; stabilizers, such as fumaric acid; coloring agents; buffering agents; dispersing agents; preservatives; organic acids; and organic bases. The aforementioned excipients are given as examples only and are not meant to include all possible choices. Additionally, many excipients may have more than one role, or be classified in more than one group; the classifications are descriptive only, and not intended to limit any use of a particular excipient.

Examples of suitable organic acids include, but are not limited to, adipic acid, ascorbic acid, citric acid, fumaric acid, malic acid, succinic acid, tartaric acid, and mixtures thereof. Suitable organic bases, include, but are not limited to, sodium citrate, sodium succinate, sodium tartrate, potassium citrate, potassium tartrate, potassium succinate, and mixtures thereof. Suitable diluents include, but are not limited to, lactose, talc, microcrystalline cellulose, sorbitol, mannitol, xylitol, fumed silica, stearic acid, magnesium stearate, sodium stearate, and mixtures thereof.

In one embodiment, a controlled release formulation of enriched (S)-bisoprolol is designed as a chronotherapeutic formulation to provide:

(i) a first phase, during which release of the drug is minimized so the plasma concentration of (S)-bisoprolol is maintained at a sub-therapeutic level in the blood stream of the subject for at least about 2 hours to about 10 hours following administration; followed by

(ii) a second phase, during which (S)-bisoprolol is released from the formulation so the plasma concentration of (S)-bisoprolol in the blood stream of the subject is maintained above a minimum therapeutic level for about 6 to about 18 hours, or for the remainder of the 24-hour period.

Such formulations provide a delay in release while the subject is sleeping (i.e., subtherapeutic level of drug) and subsequent therapeutic concentrations prior to or upon waking. The controlled release formulation may comprise a core of (S)-bisoprolol, or a pharmaceutically acceptable salt thereof, and a polymeric material.

During the first phase, the controlled release formulation of the present invention may be designed to restrict release of (S)-bisoprolol so that its plasma concentration is maintained below the therapeutically effective level. The therapeutically effective concentration may be determined empirically, by determining a subject's response and titrating a dose as necessary.

The therapeutically effective level may vary depending on the condition being treated. For example, a plasma concentration that provides a therapeutically effective level of drug for one condition may not be therapeutically effective for other conditions. As a starting point for dosing, about one-half the dose of enriched (S)-bisoprolol may be used to achieve a desired effect as compared to the dose of racemic bisoprolol required to observe the same effect.

In one embodiment, the blood plasma concentration during the first phase may be kept from about 0 to about 5 ng/ml, or any amount in between, for example, 1 ng/ml. The formulations may delay the release of therapeutic concentrations of (S)-bisoprolol for about 2 to about 10 hours, about 3 to about 8 hours, or about 3 to about 6 hours, or any hour or fraction of time in between, following administration of the formulation. For example, the present controlled release formulations may delay release of therapeutic amounts of (S)-bisoprolol for about 2, 3, 4, 5, 6, 7, 8, 9, or 10 hours, or any hour or fraction of time in between, following administration.

During the second phase, the drug is provided in an amount sufficient to exceed the minimum therapeutic level in the subject receiving the treatment. As discussed above, this therapeutic level may vary depending on the condition being treated. One of skill in the art is able to empirically measure the blood plasma concentration for a given dose, assess the resulting therapeutic effectiveness, and increase or decrease the dose as needed to achieve the desired effect. In one embodiment, the blood plasma concentration is about 2 to about 50 ng/ml, or any amount in between. This therapeutic level is maintained for the length of time necessary to achieve the desired therapeutic outcome, for example, the remainder of the 24-hour period following the first phase. Typically, (S)-bisoprolol is maintained at or above the therapeutic level for about 6 to about 22 hours, about 6 to about 18 hours, about 6 to about 12 hours, about 6 to about 15, about 8 to about 22 hours, about 8 to about 18 hours, about 8 to about 15 hours, about 8 to about 12 hours, about 10 to about 22 hours, about 10 to about 18 hours, or about 10 to about 15 hours, or any hour or fraction of time in between. Accordingly, (S)-bisoprolol is maintained at or above the therapeutic level for about 6, 7, 8, 9, 10, 11, 12, 13, 14, 15, 16, 17, 18, 19, 20, 21, or 22 hours, or any hour or fraction of time in between, measured from the end of the first phase. In this manner, the present formulations provide therapeutically effective amounts of (S)-bisoprolol throughout the day.

The controlled release formulations of the present invention may contain enriched (S)-bisoprolol or a pharmaceutically acceptable salt thereof. Examples of suitable pharmaceutically acceptable salts are listed above. In one embodiment, the salt is enriched (S)-bisoprolol fumarate or enriched (S)-bisoprolol hemifumarate (also referred to as (S)-bisoprolol fumarate 2:1).

In one embodiment, the controlled release formulations of the present invention are provided as multiparticulate formulations. Enriched (S)-bisoprolol is typically formed into an active core by applying the compound to a nonpareil seed having an average diameter in the range of about 0.4 to about 1.1 mm or about 0.85 to about 1.00 mm. The enriched (S)-bisoprolol may be applied with or without additional excipients onto the inert cores, and may be sprayed from solution or suspension using a fluidized bed coater (e.g., Wurster coating) or pan coating system. Alternatively, the enriched (S)-bisoprolol may be applied as a powder onto the inert cores using a binder to bind it onto the cores. Active cores may also be formed by extrusion of the core with suitable plasticizers (described below) and any other processing aids as necessary.

The controlled release formulations of the present invention comprise at least one polymeric material. Suitable water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof.

Suitable water-insoluble polymers include, but are not limited to, ethylcellulose, cellulose acetate cellulose propionate, cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), poly (ethylene), low density poly (ethylene), high density poly (ethylene), poly (ethylene oxide), poly (ethylene terephthalate), poly (vinyl isobutyl ether), poly (vinyl acetate), poly (vinyl chloride) or polyurethane, and/or mixtures thereof.

EUDRAGIT™ polymers (available from Rohm Pharma) are polymeric lacquer substances based on acrylates and/or methacrylates. A suitable polymer that is freely permeable to the active ingredient and water is EUDRAGIT™ RL. A suitable polymer that is slightly permeable to the active ingredient and water is EUDRAGIT™ RS. Other suitable polymers which are slightly permeable to the active ingredient and water, and exhibit a pH-dependent permeability include, but are not limited to, EUDRAGIT™ L, EUDRAGIT™ S, and EUDRAGIT™ E.

EUDRAGIT™ RL and RS are acrylic resins comprising copolymers of acrylic and methacrylic acid esters with a low content of quaternary ammonium groups. The ammonium groups are present as salts and give rise to the permeability of the lacquer films. EUDRAGIT™ RL and RS are freely permeable (RL) and slightly permeable (RS), respectively, independent of pH. The polymers swell in water and digestive juices, in a pH-independent manner. In the swollen state, they are permeable to water and to dissolved active compounds.

EUDRAGIT™ L is an anionic polymer synthesized from methacrylic acid and methacrylic acid methyl ester. It is insoluble in acids and pure water. It becomes soluble in neutral to weakly alkaline conditions. The permeability of EUDRAGIT™ L is pH dependent. Above pH 5.0, the polymer becomes increasingly permeable.

In one embodiment, the polymeric material comprises methacrylic acid co-polymers, ammonio methacrylate co-polymers, or mixtures thereof. Methacrylic acid co-polymers such as EUDRAGIT™ S and EUDRAGIT™ L (Rohm Pharma) are particularly suitable for use in the controlled release formulations of the present invention. These polymers are gastroresistant and enterosoluble polymers. The polymer films are insoluble in pure water and diluted acids. They dissolve at higher pHs, depending on their content of carboxylic acid. EUDRAGIT™ S and EUDRAGIT™ L can be used as single components in the polymer coating or in combination in any ratio. By using a combination of the polymers, the polymeric material may exhibit a solubility at a pH between the pHs at which EUDRAGIT™ L and EUDRAGIT™ S are separately soluble.

The core may comprise a polymeric material comprising a major proportion (i.e., greater than 50% of the total polymeric content) of one or more pharmaceutically acceptable water-soluble polymers, and optionally a minor proportion (i.e., less than 50% of the total polymeric content) of one or more pharmaceutically acceptable water insoluble polymers.

Alternatively, the core may comprise a polymeric material comprising a major proportion (i.e., greater than 50% of the total polymeric content) of one or more pharmaceutically acceptable water insoluble polymers, and optionally a minor proportion (i.e., less than 50% of the total polymeric content) of one or more pharmaceutically acceptable water-soluble polymers. The formulations may optionally contain a coating membrane partially or completely surrounding the core, comprising a major proportion of one or more pharmaceutically acceptable film-forming, water-insoluble polymers, and optionally a minor proportion of one or more pharmaceutically acceptable film-forming, water-soluble polymers. The water-insoluble polymer may form an insoluble matrix having a high or low permeability to (S)-bisoprolol.

In one embodiment, the polymeric material comprises methacrylic acid co-polymers, ammonio methacrylate co-polymers, or mixtures thereof. Methacrylic acid co-polymers such as Eudragit S and Eudragit L (Rohm Pharma) are particularly suitable for use in the controlled release formulations of the present invention. These polymers are gastroresistant and enterosoluble polymers. The polymer films are insoluble in pure water and diluted acids. They dissolve at higher pHs, depending on their content of carboxylic acid. Eudragit S and Eudragit L can be used as single components in the polymer coating or in combination in any ratio. By using a combination of the polymers, the polymeric material may exhibit a solubility at a pH between the pHs at which Eudragit L and Eudragit S are separately soluble.

Ammonio methacrylate co-polymers such as Eudragit RS and Eudragit RL (Rohm Pharma) are also particularly suitable for use in the controlled release formulations of the present invention. These polymers are insoluble in pure water, dilute acids, buffer solutions, or digestive fluids over the entire physiological pH range. The polymers swell in water (and digestive fluids independently of pH). In the swollen state they are then permeable to water and dissolved actives. The permeability of the polymers depends on the ratio of ethylacrylate (EA), methyl methacrylate (MMA), and trimethylammonioethyl methacrylate chloride (TAMCI) groups in the polymer. Those polymers having EA:MMA:TAMCI ratios of 1:2:0.2 (Eudragit RL) are more permeable than those with ratios of 1:2:0.1 (Eudragit RS). Polymers of Eudragit RL are insoluble polymers of high permeability. Polymers of Eudragit RS are insoluble films of low permeability.

The ammonio methacrylate co-polymers may be combined in any desired ratio. For example, the ratio of Eudragit RS:Eudragit RL (90:10) may be used. The ratios may be adjusted to provide a delay in release of the drug. For example, the ratio of Eudragit RS:Eudragit RL may be about 100:0 to about 80:20, about 100:0 to about 90:10, or any ratio in between. In such formulations, the less permeable polymer Eudragit RS would generally comprise the majority of the polymeric material.

The ammonio methacrylate co-polymers may be combined with the methacrylic acid co-polymers within the polymeric material in order to achieve the desired delay in release of the drug. Ratios of ammonio methacrylate co-polymer (e.g., Eudragit RS) to methacrylic acid co-polymer in the range of about 99:1 to about 20:80 may be used. The two types of polymers can also be combined into the same polymeric material, or provided as separate coats that are applied to the core.

In addition to the Eudragit polymers described above, a number of other such copolymers may be used to create a delay in drug release. These include methacrylate ester co-polymers (e.g., Eudragit NE 30D). Further information on the Eudragit polymers is to be found in “Chemistry and Application Properties of Polymethacrylate Coating Systems”, in Aqueous Polymeric Coatings for Pharmaceutical Dosage Forms, ed. James McGinity, Marcel Dekker Inc., New York, pg 109-114).

The polymeric material typically comprises one or more soluble excipients so as to increase the permeability of the polymeric material. Suitably, the soluble excipient is selected from among a soluble polymer, a surfactant, an alkali metal salt, an organic acid, a sugar, and a sugar alcohol. Such soluble excipients include polyvinyl pyrrolidone, polyethylene glycol, sodium chloride, surfactants such as sodium lauryl sulfate and polysorbates, organic acids such as acetic acid, adipic acid, citric acid, fumaric acid, glutaric acid, malic acid, succinic acid, and tartaric acid and sugars such as dextrose, fructose, glucose, lactose and sucrose, and sugar alcohols such as lactitol, maltitol, mannitol, sorbitol and xylitol, xanthan gum, dextrins, and maltodextrins. In some particular embodiments, polyvinyl pyrrolidone, mannitol and/or polyethylene glycol are the soluble excipients. The soluble excipient is typically used in an amount of from about 1% to about 10% by weight, based on the total dry weight of the polymer.

The polymeric material can also include one or more auxiliary agents such as a filler, a plasticizer and/or an anti-foaming agent. Representative fillers include talc, fumed silica, glyceryl monostearate, magnesium stearate, calcium stearate, kaolin, colloidal silica, gypsum, micronized silica and magnesium trisilicate. The quantity of filler used typically ranges from about 2% to about 300% by weight, and can range from about 20 to about 100%, based on the total dry weight of the polymer. In one embodiment, talc is the filler.

The coatings can also include a material that improves the processing of the polymers. Such materials are generally referred to as plasticizers and include, for example, adipates, azelates, benzoates, citrates, isoebucates, phthalates, sebacates, stearates and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In one embodiment, the plasticizer is dibutyl sebacate. The amount of plasticizer used in the polymeric material typically ranges from about 10% to about 50%, for example, about 10, 20, 30, 40, or 50%, based on the weight of the dry polymer.

In one embodiment, the anti-foaming agent is simethicone. The amount of anti-foaming agent used typically comprises from about 0% to about 0.5% of the final formulation.

The amount of polymer to be used in controlled release formulations is typically adjusted to achieve the desired drug delivery properties, including the amount of drug to be delivered, that rate and location of drug delivery, the time delay of drug release, and the size of the multiparticulates in the formulation. The amount of polymer applied typically provides about a 10 to about 100% weight gain to the cores. In one embodiment, the weight gain from the polymeric material is about 25 to about 70%.

The combination of all solid components of the polymeric material, including co-polymers, fillers, plasticizers, and optional excipients and processing aids, typically provides an about 10 to about 450% weight gain on the cores. In one embodiment, the weight gain is about 30 to about 160%.

The polymeric material can be applied by any known method, for example, by spraying using a fluidized bed coater (e.g., Wurster coating) or pan coating system.

The coated cores are typically dried or cured after application of the polymeric material. Curing means that the multiparticulates are held at a controlled temperature for a time sufficient to provide stable release rates. Curing can be performed for example in an oven or in a fluid bed drier. Curing can be carried out at any temperature above room temperature.

A sealant or barrier can be applied to the polymeric coating. A sealant or barrier layer may also be applied to the core prior to applying the polymeric material. The sealant or barrier layer does not modify the release of enriched (S)-bisoprolol significantly. Suitable sealants or barriers are permeable or soluble agents such as hydroxypropyl methylcellulose, hydroxypropyl cellulose, hydroxypropyl ethylcellulose, and xanthan gum. Hydroxypropyl methylcellulose is particularly useful in this regard.

Other agents can be added to improve the processability of the sealant or barrier layer. Such agents include talc, colloidal silica, polyvinyl alcohol, titanium dioxide, micronized silica, fumed silica, glycerol monostearate, magnesium trisilicate or magnesium stearate or a mixture thereof. The sealant or barrier layer can be applied from solution (e.g., aqueous) or suspension using any known means, such as a fluidized bed coater (e.g., Wurster coating) or pan coating system. Suitable sealants or barriers include, for example, OPADRY WHITE Y-1-7000 and OPADRY OY/B/28920 WHITE, each of which is available from Colorcon Limited, England.

The invention also provides an oral dosage form containing a multiparticulate, enriched (S)-bisoprolol formulation as hereinabove defined, in the form of caplets, capsules, particles for suspension prior to dosing, sachets, or tablets. When the dosage form is in the form of tablets, the tablets may be, for example, disintegrating tablets, fast dissolving tablets, effervescent tablets, fast melt tablets, and/or mini-tablets. The dosage form can be of any shape suitable for oral administration of a drug, such as spheroidal, cube-shaped oval, or ellipsoidal. The dosage forms will be prepared from the multiparticulates in a manner known in the art and include addition pharmaceutically acceptable excipients, as desired.

The thickness of the polymer in the formulations, the amounts and types of polymers, and the ratio of water-soluble polymers to water-insoluble polymers in the controlled release formulations are generally selected to achieve a desired release profile of (S)-bisoprolol. For example, by increasing the amount of water insoluble-polymer relative to the water soluble-polymer, the release of the drug may be delayed or slowed.

In one embodiment, the enriched (S)-bisoprolol formulations initially delay the release of the drug. Following the delay, the formulation may rapidly release the drug, or optionally, control the release for a specified period of time. The controlled release over time is useful, for example, to provide a subject with therapeutic drug levels in the morning hours following a nighttime administration. As a result, a subject can take the drug at night prior to sleep, and obtain the therapeutic benefits in the morning hours. This is particularly useful in treating, preventing, and/or managing morning pathologies observed in subjects suffering from cardiovascular conditions.

Alternatively, the formulations may release (S)-bisoprolol soon after administration. Such formulations would provide a rapid and/or immediate therapeutic effect for the subject. These formulations may control release of the drug over a specified period, or alternatively, rapidly release the drug.

In one embodiment, enriched (S)-bisoprolol is provided in a controlled release formulation designed to provide an in vitro dissolution profile substantially corresponding to the following, when measured by U.S. Pharmacopoeia (USP) Type 1 Apparatus (baskets) at 37° C. and 50 rpm or higher in 0.01 N HCl for the first 2 hours, followed by transfer to phosphate buffer at pH 6.8 or higher for the remainder of the measuring period:

(a) from about 0% to about 10% of the total (S)-bisoprolol is released after 2 hours;

(b) less than about 50% of the total (S)-bisoprolol is released after 4 hours; and

(c) greater than about 50% of the total (S)-bisoprolol is released after 22 hours of measurement in said apparatus.

One of skill in the art is familiar with the techniques used to determine other such dissolution profiles. The standard methodologies set forth in the U.S. Pharmacopeia, which is incorporated herein by reference for this purpose, may be used. For example, the dissolution profile may be measured in either a U.S. Pharmacopeia (USP) Type 1 Apparatus (baskets) or a U.S. Pharmacopeia (USP) Type 2 Apparatus (paddles). For pH-independent formulations, the formulations may be tested in phosphate buffer at pH 6.8, 37° C., and 50 rpm or higher. For pH-dependent formulations, the formulations may be tested in 0.01 N HCl for the first 2 hours at 37° C. and 50 rpm or higher, followed by transfer to phosphate buffer at pH 6.8 or higher for the remainder of the test. Other buffer systems suitable for measuring the dissolution profile for pH-dependent and pH-independent formulations are well-known to those of skill in the art.

In some embodiments, the dissolution profile of the enriched (S)-bisoprolol formulations may substantially mimic one or more of the profiles provided below, when measured by USP Type 1 Apparatus (baskets) at 37° C. and 100 rpm in 0.01 N HCl for the first 2 hours, followed by transfer to phosphate buffer at pH 7.2 for the remainder of the measuring period:

| Time (hours) | % Released |
|---|---|
| 0-2 | <10% |
| 4 | <30% |
| 6 | 10-40% |
| 8 | 30-70% |
| 12 | >80% |

The amount of the dose administered, as well as the dose frequency, will vary depending on the particular dosage form used and route of administration. The amount and frequency of administration will also vary according to the age, body weight, and response of the individual subject. A competent physician can readily determine typical dosing regimens without undue experimentation. It is also noted that the clinician or treating physician will know how and when to interrupt, adjust, or terminate therapy in conjunction with individual subject response.

In general, the total daily dosage for treating, preventing, and/or managing the cardiovascular conditions described herein is from about 0.1 mg to about 100 mg enriched (S)-bisoprolol, or a pharmaceutically acceptable salt thereof. The daily dose may range from about 0.1 mg to about 100 mg, or from about 0.5 mg to about 75 mg, or from about 1 mg to about 60 mg, or from about 1 mg to about 50 mg, or from about 1 mg to about 30 mg, or from about 1 mg to about 25 mg, or from about 1 mg to about 20 mg, or from about 2 mg to about 20 mg, or from about 1 mg to about 10 mg, or from about 2 mg to about 10 mg. A single dose may be formulated to contain about 0.1, 0.2, 0.3, 0.4, 0.5, 0.75, 1, 1.25, 1.5, 2, 2.5, 3, 3.5, 3.75, 4, 4.5, 5, 5, 6, 7, 7.5, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 mg of enriched (S)-bisoprolol. In one embodiment, enriched (S)-bisoprolol, or pharmaceutically acceptable salt thereof comprises about 0.5 to about 20%, about 0.5 to about 8%, or about 0.5 to about 4% of the total weight of the formulation.

The pharmaceutical compositions containing enriched (S)-bisoprolol may be administered in single or divided doses 1, 2, 3, 4, or more times each day. Alternatively, the dose may be delivered once every 2, 3, 4, 5, or more days. In one embodiment, the pharmaceutical compositions are administered once per day.

Any of the pharmaceutical compositions and dosage forms described herein may further comprise one or more pharmaceutically active compounds other than enriched (S)-bisoprolol. Such compounds may be included to treat, prevent, and/or manage the same condition being treated, prevented, and/or managed with enriched (S)-bisoprolol, or a different one. For example, those of skill in the art are familiar with examples of the techniques for incorporating additional active ingredients into compositions comprising enriched (S)-bisoprolol. Alternatively, such additional pharmaceutical compounds may be provided in a separate formulation and co-administered to a subject along with an enriched (S)-bisoprolol composition according to the present invention. Such separate formulations may be administered before, after, or simultaneously with the administration of the enriched (S)-bisoprolol compositions of the present invention. In one embodiment, the enriched (S)-bisoprolol formulation comprises and/or is co-administered with one or more other compounds including, but not limited to: diuretics, in particular, thiazide diuretics (e.g., hydrochlorothiazide); inotropic agents; antiplatelet agents; statins; vasodilators; ACE inhibitors; angiotensin receptor inhibitors; calcium channel blockers (also known as calcium antagonists); and/or nitrates.

The invention is further illustrated by reference to the following examples. It will be apparent to those skilled in the art that many modifications, both to the materials and methods, may be practiced without departing from the purpose and scope of the invention.

### EXAMPLES

### Example 1

### Preparation of Rapid Release Multiparticulate Formulations Containing Eneriched (S)-bisoprolol

A solution of enriched (S)-bisoprolol fumarate 2:1 is prepared as follows: 180 g of enriched (S)-bisoprolol fumarate 2:1 is added to 529.2 g of purified water; the mixture is stirred for 10 minutes to dissolve the drug; 10.8 g of talc USP (Whitaker, Clark and Daniels Inc., South Plainfield, N.J., USA) are added to the solution and the mixture is stirred for 20 min. The resulting suspension is sprayed onto 0.85-1.00 mm non pareil seeds (NP Pharma SA, France) in a fluid bed apparatus (GPCG-3, Glatt) using Wurster coating. The drug is layered onto the non pareil seeds to give a 5% drug weight gain. The spray rate for drug layering is 1.5-3.6 g/min/kg, the inlet temperature is 50° C. and the non-pareils are maintained at 37-42° C. The drug-loaded rapid release multiparticulates are cooled in a Glatt GPCG-3 for 10 minutes. The multiparticulates are screened to remove oversized beads and fine material.

### Example 2

### Preparation of Eudragit RS:RL (90:10) Coated Multiparticulates Containing Enriched (S)-bisoprolol Fumarate 2:1

A Eudragit RS:Eudragit RL (90:10) aqueous dispersion is prepared as follows: 0.5 g of Simethicone emulsion USP (OSI Specialties, Belgium) and 300 g of talc USP are added with mixing to 1139.5 g of purified water; the mixture is stirred for 15 minutes; 900 g of Eudragit RS 30D and 100 g of Eudragit RL 30D (ammonio methacrylate co-polymers in the form of aqueous dispersions from Rohm Pharma, Germany) are added to the mixture and stirred for 20 minutes; 60 g of dibutyl sebacate (Morflex Inc., Greensboro, N.C., USA) are added to the mixture and stirred for 20 minutes. The aqueous dispersion is screened through a 500 μm sieve.

The resulting dispersion is sprayed onto rapid release multiparticulates prepared according to Example 1, using a fluid bed apparatus as used in Example 1. Spray rate is 3-10 g/min/kg, and the inlet temperature is 45-50° C. The rapid release multiparticulates are maintained at 30-40° C. and the air volume is 150-190 m³/h. A polymer coating of 40% polymer weight gain is coated onto the rapid release multiparticulates. The coated multiparticulates are cooled in the Glatt GPCG-3 for 30 minutes post coating, then dried/cured in the following manner:

Phase 1: Temperature is 50° C. for 11 h. 25 min., then drops (steamer down) to a low of 40° C. over 1 h. 35 min.; temperature fluctuates between 40-56° C. for 30 min; then remains at 50° C. for a further 34 h. 30 min;

Phase 2: Temperature drops to 35° C. over 2 h. 25 min.; then remains at 35° C. for a further 29 h. 9 min.

The multiparticulates are screened to remove oversized multiparticulates and fine material.

### Example 3

### Preparation of Eudragit RS:Eudragit RL (90:10) Coated Multiparticulates Containing Enriched (S)-bisoprolol Fumarate 2:1

Bisoprolol rapid release multiparticulates (prepared according to Example 1) are coated with the Eudragit RS:Eudragit RL (90:10) aqueous dispersions (prepared according to Example 2) to achieve a weight gain of 50%. The multiparticulates are cured in the following manner: temperature is 50° C. for 8 h. 45 min., then drops (steamer down) to a low of 30° C. over 5 h. 55 min. Oven is reset and remains at 50° C. for a further 24 h. 25 min. Oven is reset again (to account for loss of hours when steamer down). The temperature fluctuates between 38° C. and 53° C. for 1 h. 45 min; and then remains at 50° C. for 15 h. 38 min. Temperature ramps down to 29° C. over the next 5 hr. 30 min. The multiparticulates are screened as described in Example 2.

### Example 4

### Preparation of Eudragit L Multiparticulates Containing Enriched (S)-bisoprolol Fumarate 2:1

Enriched (S)-Bisoprolol rapid release multiparticulates (prepared according to Example 1) are coated with Eudragit RS:Eudragit RL (90:10) aqueous dispersion (prepared according to Example 2) to a polymer weight gain of 30%. The multiparticulates are cured and screened as described in Example 2.

A Eudragit L polymer solution is then prepared as follows: 120 g of purified water, 1705 g of isopropyl alcohol and 50 g of dibutyl sebacate are mixed together and stirred for 10 minutes; 125 g of talc USP is added to the mixture and stirred for 15 minutes; 2000 g of Eudragit L 12.5 (solution of methacrylate co-polymer from Rohm Pharma, Germany) is added and stirred for 15 minutes.

The resulting polymer solution is sprayed onto the enriched (S)-bisoprolol multiparticulates coated to 30% polymer weight gain with the Eudragit RS:Eudragit RL (90:10) polymer coat described above. The Eudragit L solution is then applied with a fluid bed apparatus (Glatt GPCG-3) using Wurster coating. Spray rate is 6-16 g/min/kg, and the inlet temperature is 35-40° C. The multiparticulates are maintained at 30-32° C. during coating and the air volume is 120-140 m³/h. A polymer coating of 20% Eudragit L weight gain is coated onto the Eudragit RS:Eudragit RL (90:10) coated multiparticulates. The Eudragit L coated multiparticulates are cured in the Glatt GPCG-3 for 60 minutes post coating. The multiparticulates are screened to remove oversized multiparticulates and fine material.

### Example 5

### Preparation of Eudragit S Coated Multiparticulates Containing Enriched (S)-bisoprolol Fumarate 2:1

A Eudragit S solution is prepared as follows: 300 g of purified water and 4262.5 g of isopropyl alcohol are stirred together for 5 min.; 125 g of dibutyl sebacate is added and the mixture stirred for 5 min.; 312.5 g of talc USP are added to the mixture and stirred for 15 minutes; 5000 g of Eudragit S 12.5 (solution of methacrylate copolymer from Rohm Pharma, Germany) is added and stirred for 30 min. The resulting solution is sprayed onto rapid release multiparticulates prepared according to Example 1, using a fluid bed apparatus as used in Example 1. Spray rate is 3-12 g/min/kg, and the inlet temperature is 38-40° C. The rapid release multiparticulates are maintained at 30-35° C. and the air volume is 130-160 m³/hr. A polymer coating of 35% polymer weight gain is coated onto the rapid release multiparticulates. The coated multiparticulates are cooled in the Glatt GPCG-3 for 10 minutes post coating, then dried/cured in the following manner:

15 h. at 40° C.; cool to 34° C. over 1 h. 45 min.; keep at 34-35° C. for 7h. 15 min. The multiparticulates are screened to remove oversized multiparticulates and fine material.

### Example 6

### In Vitro Dissolution of Multiparticulates

The dissolution profile for the multiparticulates produced as described in Examples 2 to 5 above are determined. Multiparticulates manufactured as described in Examples 2 and 3 are tested in phosphate buffer, pH 6.8, in an USP Type 2 apparatus, using paddles, at 50 rpm. Multiparticulates manufactured as described in Examples 4 and 5 are tested in 0.01 N HCl for 2 h, then transferred to phosphate buffer pH 6.8 or higher for the remainder of the testing interval. An USP Type 1 apparatus with baskets is used at 100 rpm, rather than paddles as in the formulations of Examples 4 and 5.

In addition to the formulations described in Examples 2 to 6 above, different polymer coating combinations are coated onto rapid release multiparticulates manufactured as described in Example 1, above, using the GPCG-3 or the Uni-Glatt. Examples of the additional formulations manufactured are described below.

### Example 7

### Preparation of Eudragit RS:Eudragit RL (97.5:2.5) Coated Multiparticulates Containing Enriched (S)-bisoprolol Fumarate 2:1

A Eudragit RS:Eudragit RL (97.5:2.5) aqueous dispersion is prepared as follows: 0.8 g of Simethicone emulsion USP (OSI Specialties, Belgium) and 480 g of talc are added with mixing to 1823.2 g of purified water. The mixture is stirred for 15 minutes; 1560 g of Eudragit RS 30D and 40 g of Eudragit RL 30D (ammonio methacrylate co-polymers in the form of aqueous dispersions from Rohm Pharma, Germany) are added to the mixture and stirred for 10 minutes; 96 g of dibutyl sebacate (Morflex Inc., Greensboro, N.C. USA) are added to the mixture and stirred for 15 minutes.

The resulting combined dispersion is sprayed onto rapid release multiparticulates prepared according to Example 1, but manufactured using a fluid bed apparatus (Uni-Glatt) and Wurster coating. Spray rate is 3.2-8.6 g/min/kg, and the inlet temperature is 34-46° C. The outlet air flap setting on the Uni-Glatt is maintained at a setting of 50. A polymer coating of 20% polymer weight gain is coated onto the rapid release multiparticulates. The coated multiparticulates are cooled in the Uni-Glatt for 30 minutes post coating, then dried/cured in an oven at 50° C. for 86 h. The multiparticulates are screened to remove oversized multiparticulates and fine material.

### Example 8

### Preparation of Eudragit RS:Eudragit RL (95:5) Coated Multiparticulates Containing Enriched (S)-bisoprolol Fumarate 2:1

A Eudragit RS:Eudragit RL (95:5) aqueous dispersion is prepared as follows: 0.6 g of Simethicone emulsion USP (OSI Specialties, Belgium) and 360 g of talc are added with mixing to 1367.4 g of purified water; the mixture is stirred for 15 minutes; 1140 g of Eudragit RS 30D and 60 g of Eudragit RL 30D (ammonio methacrylate co-polymers in the form of aqueous dispersions from Rohm Pharma, Germany) are added to the mixture and stirred for 10 min.; 72 g of dibutyl sebacate (Morflex Inc., Greensboro, N.C., USA) are added to the mixture and stirred for 15 minutes.

The resulting dispersion is sprayed onto rapid release multiparticulates prepared according to Example 1, but manufactured using a fluid bed apparatus (Uni-Glatt) using Wurster coating. Spray rate is 1.4-10.7 g/min/kg, and the inlet temperature is 38-52° C. The outlet air flap setting on the Uni-Glatt is maintained at a setting of 50. A polymer coating of 20% polymer weight gain is coated onto the rapid release multiparticulates. The coated multiparticulates are cooled in the Uni-Glatt for 30 minutes post coating, then dried/cured in an oven at 50° C. for 48 h. The multiparticulates are screened to remove oversized multi particulates and fine material.

### Example 9

### Preparation of Eudragit RS:PVP K-30 (95:5) Coated Multiparticulates Containing Enriched (S)-bisoprolol Fumarate 21

A Eudragit RS:PVP K-30 (95:5) aqueous dispersion is prepared as follows: 0.6 g of Simethicone emulsion USP (OSI Specialities, Belgium) and 18 g of Kollidon 30 (BASF) are added with mixing to 1409.4 g of purified water; the mixture is stirred for 10 minutes; 360 g of talc USP are added to the mixture and stirred for 15 minutes; 1140 g of Eudragit RS 30D (ammonio methacrylate co-polymer in the form of aqueous dispersion from Rohm Pharma, Germany) is added to the mixture and stirred for 10 minutes; 72 g of dibutyl sebacate (Morflex Inc., Greensboro, N.C., USA) are added to the mixture and stirred for 15 minutes.

The resulting dispersion is sprayed onto rapid release multiparticulates prepared according to Example 1, using a fluid bed apparatus as used in Example 1. Spray rate is 4.2-15.2 g/min/kg, and the inlet temperature is 48-54° C. The rapid release multiparticulates are maintained at 37-44° C. and the air volume is 147-231 m³/h. A polymer coating of 20% polymer weight gain is coated onto the rapid release multiparticulates. The coated multiparticulates are cooled in the Glatt GPCG-3 for 30 minutes post coating, then dried/cured in an oven at 50° C. for 46 h. The multiparticulates are screened to remove oversized multiparticulates and fine material.

### Example 10

### In Vitro Dissolution of Multiparticulates

Dissolution profiles for the multiparticulates produced as described in Examples 7 to 9 are determined. Tests are conducted in phosphate buffer, pH 6.8, using a USP Type 2 apparatus (paddles) at 50 rpm.

### Example 11

### Preparation of Multiparticulates Containing Enriched (S)-bisoprolol and a Sealant

A solution of enriched (S)-bisoprolol is prepared as follows: 180 g of enriched (S)-bisoprolol fumarate 2:1 are added to 529.2 g of purified water, and stirred for 10 minutes to dissolve the drug; 10.8 g of talc USP (Whitaker, Clark and Daniels Inc., South Plainfield, N.J., USA) are added to the solution and the mixture is stirred for 20 minutes.

The suspension is sprayed onto 0.85-1.00 mm non-pareil seeds (NP Pharma SA, France) in a fluid bed apparatus (GPCG-3, Glatt) using Wurster coating. The drug is layered onto the non-pareil seeds to give a 5% drug weight gain. The spray rate for drug layering is 1.5-3.6 g/min/kg, the inlet temperature is 50° C., and the non-pareil seeds are maintained at 37-42° C. The drug loaded rapid release multiparticulates are cooled in the Glatt GPCG-3 for 10 minutes. The multiparticulates are screened to remove oversized beads and fine material.

A sealant is then coated onto the rapid release multiparticulates. A suspension of Opadry White in water is prepared as follows: 100 g of Opadry White Y-1-7000 (Colorcon Ltd., England) are added to 900 g of purified water with stirring; the mixture is stirred for a further 45 minutes to disperse the Opadry White. The suspension is screened through a 500 μam screen and sprayed onto the rapid release enriched (S)-bisoprolol multiparticulates (manufactured as described above) in a fluid bed apparatus (GPCG-3, Glatt) using Wurster coating. The Opadry White is layered onto 2.2 Kg of rapid release multiparticulates to give a 2% solids weight gain. The spray rate for coating with the Opadry White suspension is 1.7-2.5 g/min/kg, the inlet temperature is 44-47° C. and the non-pareil seeds are maintained at 36-39° C. The Opadry coated multiparticulates are cooled in the Glatt GPCG-3 for 10 minutes. The multiparticulates are screened to remove oversized beads and fine material.

The sealant coated multiparticulates are then coated with a preparation of Eudragit RS:Eudragit RL (90:10) coated. A Eudragit RS-Eudragit RL (90:10) aqueous dispersion is prepared as follows: 1.62 of Simethicone emulsion USP (OSI Specialties, Belgium) and 960 g of talc USP are added with mixing to 3646.4 g of purified water; the mixture is stirred for 15 minutes; 2880 g of Eudragit RS 30D and 320 g of Eudragit RL 30D (ammonic methacrylate co-polymers in the form of aqueous dispersions from Rohm Pharma, Germany) are added to the mixture and stirred for 20 minutes; 192 g of dibutyl sebacate (Morflex Inc., Greensboro, N.C., USA) are added to the mixture and stirred for 20 minutes. The aqueous dispersion is screened through a 500 μm sieve.

The resulting dispersion is sprayed onto the 2% Opadry White coated multiparticulates prepared above using a fluid bed apparatus. Spray rate is 2.7-10.9 g/min/kg, and the inlet temperature is 45-48° C. The Opadry white coated multiparticulates are maintained at 28-40° C. and the air volume is 149-169 m³/hr. A polymer coating of 30% polymer weight gain is coated onto the Opadry White multiparticulates. The coated multiparticulates are cooled in the Glatt GPCG-3 for 30 minutes post coating. A further 10.65% polymer weight gain is achieved by continuing the coating of the Eudragit RS:Eudragit RL (90:10) aqueous dispersion onto 1 Kg the 30% polymer coated multiparticulates in the Glatt GPCG-3. Spray rate is 7.7-11.3 g/min/kg, and the inlet temperature is 46-49° C. The 30% polymer coated multiparticulates are maintained at 32.9-40.4° C. and the air volume is 126-136 m³/hr. The total polymer coating applied to the Opadry White coated multiparticulates at the end of this process is 40% polymer weight gain. The coated multiparticulates are cooled in the Glatt GPCG-3 for 30 minutes post coating. The coated multiparticulates are dried/cured in an oven at the following temperatures and times:

Phase 1: 50° C. for 33 hr 25 minutes, then temperature drops to a low of 32° C. for 2 hours; temperature returns to 50° C. for a further 12 h. 35 min.; then drops to 35° C. over 30 hours 45 minutes.

The multiparticulates are screened to remove oversized multiparticulates and fine material. The dissolution profile for this formulation is tested in phosphate buffer, pH 6.8, using USP Type 2 apparatus withpaddles, at 50 rpm.

### Example 12

### Preparation of Capsules Containing 2.5 mg of Enriched (S)-bisoprolol Fumarate 2:1

Coated multiparticulates manufactured as described in Examples 2-5 are encapsulated in size 2 hard gelatin capsules to give dosage forms containing the equivalent of 2.5 mg of enriched (S)-bisoprolol fumarate 2:1. The dissolution profiles for the encapsulated formulations are then determined. The encapsulated formulations of examples 2 and 3 are tested in phosphate buffer, pH 6.8, in a USP Type 2 apparatus (paddles) at 50 rpm. The encapsulated formulations of examples 4 and 5 are tested in an USP Type 1 apparatus (baskets at 100 RPM) in 0.01 N HCl for the first 2 hours, then transferred to phosphate buffer, pH 6.8 or higher, for the remainder of the testing period. All tests are performed at 37° C.

### Example 13

### Biostudy

An open label, single dose, five treatment, five period, balanced, randomized crossover study is designed to compare the bioavailability of the formulations described in Examples 2-5, as encapsulated according to Example 12 (2.5 mg of enriched (S)-bisoprolol fumarate 2:1) relative to a reference formulation comprising racemic bisoprolol, such as Concor (Merck, 5 mg). Fifteen healthy male volunteers are dosed as one group, with each volunteer being dosed on five occasions with at least a seven-day washout period between each dose. The volunteers are fasting from food and beverages other than water for at least four hours prior to dosing in each treatment period, and water is proscribed one hour before and one hour after dosing. The volunteers are fed an evening meal (approximately 17:00 hours);

dosing occurs at night (approximately 22.00 hours), followed by at least a 10-hour fast. Venous blood specimens are obtained from the volunteers at regular time intervals following each dosing.

The mean plasma concentrations for (S)-bisoprolol for the volunteers are determined and compared with conventional formulations of racemic bisoprolol.

### Example 14

### Comparison of Racemic Bisoprolol and Enriched (R)-bisoprolol with Enriched (S)-bisoprolol

To compare the cardiovascular effects of racemic, enriched (R)-, and enriched (S)-bisoprolol, an exercise test is conducted. Healthy subjects are selected and divided into four treatment groups. Subjects in group 1 are to be given a dose of enriched (S)-bisprolol ranging from 0.5 to 50 mg; subjects in group 2 are to be given a dose of enriched (R)-bisoprolol ranging from 0.5 to 50 mg; subjects in group 3 are to be given a dose of racemic bisoprolol ranging from 0.5 to 50 mg; subjects in group 4 are to be given a placebo.

The test subjects perform an exercise routine: each subject steps on and off a 40 to 50 cm high box, at a rate of 30 steps per minute, for 3 to 5 minutes. Within 5 seconds of completing the routine, a first heart rate (A) is measured. After a resting period of 1-2 hours, the subjects are treated with bisoprolol or placebo, depending on their group. After a second resting period of 2-3 hours, the subjects perform the identical exercise routine again. Within 5 seconds of completing the routine, a second heart rate (B) is measured.

The percent heart rate inhibition achieved by the various amounts of drugs administered is calculated as 100*[A-B]/A. Dose response curves are prepared and compared.

### Example 15

### Use of Enriched (S)-Bisoprolol Oral Dosage Form to Treat a Subject Suffering from Hypertension

Enriched (S)-bisoprolol formulations are prepared, as described in Examples 2-5. A subject diagnosed with essential hypertension and desiring a lowering of their blood pressure receives a daily administration of enriched (S)-bisoprolol formulation containing about 2.5 mg of the drug. The subject's blood pressure is monitored to assess the effect of the 2.5 mg dose on blood pressure for about 1 to about 4 weeks. Typically, an initial rapid drop in blood pressure is observed, followed by a secondary drop in blood pressure. Once the effect of the 2.5 mg dose is established, the dose can be safely titrated by increasing the amount of enriched (S)-bisoprolol over several days or weeks to higher levels that achieve the desired drop in blood pressure.

The formulations of this example, which comprise less than the amount of drug used in conventional racemic formulations, achieve an equivalent or better therapeutic effect, while exhibiting fewer side effects.

### Example 16

### Use of Enriched (S)-Bisoprolol Oral Dosage Form to Treat a Subject Suffering from Angina Pectoris

Enriched (S)-bisoprolol formulations are prepared, as described in Examples 2-5. A subject diagnosed with angina pectoris, having a positive symptom-limited exercise tolerance test, and desiring a relief of their symptoms receives a daily administration of enriched (S)-bisoprolol formulation containing about 5 mg of the drug. The subject's exercise tolerance is monitored to assess the effect of the 5 mg dose after about 4 weeks. Typically exercise time and time to 1 mm ST-segment depression is increased. Once the effect of the 5 mg dose is established, the dose can be safely titrated by increasing or decreasing the amount of enriched (S)-bisoprolol over several days or weeks to levels that achieve the desired relief from the symptoms of angina pectoris.

The formulations of this example, which comprise less than the amount of drug used in conventional racemic formulations, achieve an equivalent or better therapeutic effect, while exhibiting fewer side effects.

What is claimed is:

1. A method of treating one or more cardiovascular conditions comprising administering a therapeutically effective amount of enriched (S)-bisoprolol, or a pharmaceutically acceptable salt thereofto a subject in need of such treatment, wherein the administration of the enriched (S)-bisoprolol, or pharmaceutically acceptable salt thereof, reduces one or more side-effects relative to administration of the same amount of a racemic mixture of bisoprolol.

2. The method of claim 1, wherein the cardiovascular condition is chosen from hypertension, ischemic heart disease, atrial fibrillation, congestive heart failure, angina pectoris, and cardiac arrhythmia.

3. The method of claim 1, wherein the cardiovascular condition is chosen from congestive heart failure, angina pectoris, and hypertension.

4. The method of claim 1, wherein the enriched (S)-bisoprolol is provided in a pharmaceutical formulation.

5. The method of claim 4, wherein the enriched (S)-bisoprolol formulation is a solid dosage form.

6. The method of claim 5, wherein the solid dosage form comprises at least one system chosen from diffusion-controlled, matrix-type, osmotic-controlled, and ionic exchange systems.

7. The method of claim 6, wherein the solid dosage form comprises a diffusion-controlled system.

8. The method of claim 7, wherein the diffusion-controlled system comprises at least one polymer chosen from water-soluble polymers and water-insoluble polymers.

9. The method of claim 8, wherein the at least one polymer exhibits a pH-dependent solubility.

10. The method of claim 5, wherein the solid dosage form comprises a matrix-type dosage form, which comprises at least one polymer chosen from water-soluble polymers and water-insoluble polymers.

11. The method of claim 10, wherein the dosage form exhibits a mechanism of release based upon at least one of diffusion and erosion.

12. The method of claim 5, wherein the solid dosage form comprises an osmotic-type system.

13. The method of claim 12, wherein the osmotic-type system comprises a selectively permeable membrane.

14. The method of claim 1, wherein the amount of enriched (S)-bisoprolol administered ranges from about 0.1 mg to about 50 mg.

15. The method of claim 14, wherein the amount of enriched (S)-bisoprolol administered ranges from about 1 mg to about 20 mg.

16. The method of claim 1, wherein enriched (S)-bisoprolol is administered in combination with one or more additional pharmaceutically active compounds.

17. The method of claim 5, wherein the dosage form is a controlled-release dosage form.

18. The method of claim 17, wherein the controlled-release formulation comprises a coating including one or more polymers.

19. The method of claim 18, wherein the one or more polymers are chosen from water-soluble polymers, water-insoluble polymers, and combinations thereof.

20. The method of claim 19, wherein the water soluble polymer is chosen from polyvinyl alcohol, polyvinylpyrrolidone, methyl cellulose, hydroxypropyl cellulose, hydroxypropyl methyl cellulose, polyethylene glycol, and mixtures thereof.

21. The method of claim 19, wherein the water insoluble polymer is chosen from ethylcellulose, cellulose acetate, cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene), poly(propylene), poly(ethylene oxide), poly(ethylene terephthalate), poly(vinyl isobutyl ether), poly(vinyl acetate), poly(vinyl chloride), polyurethane, and mixtures thereof.

22. A composition comprising a therapeutically effective amount of enriched (S)-bisoprolol, or a pharmaceutically acceptable salt thereof, and one or more pharmaceutically acceptable excipients.

23. The composition of claim 22, comprising about 0.1, 0.5, 1, 1.25, 2, 2.5, 3, 3.75, 4, 5, 7.5, 10, or 15 mg of enriched (S)-bisoprolol.

24. The composition of claim 23, comprising about 1.25, 2.5, 5, or 7.5 mg of enriched (S)-bisoprolol.

25. The composition of claim 22, wherein the composition is a solid dosage form.

26. The composition of claim 22, wherein the composition is suitable for oral, nasal, parenteral, intracisternal, buccal, sublingual or topical administration.

27. The composition of claim 26, wherein the composition is suitable for oral administration.

28. The composition of claim 27, wherein the composition is provided as a tablet, sachet, caplet, or capsule.

29. The composition of claim 22, wherein the one or more excipients are chosen from a starch, sugar, cellulose, diluent, granulating agent, lubricant, binder, disintegrating agent, wetting agent, emulsifier, coloring agent, release agent, coating agent, sweetening agent, flavoring agent, perfuming agent, preservative, antioxidant, plasticizer, gelling agent, thickener, hardener, setting agent, suspending agent, surfactant, humectant, carrier, stabilizer, and combinations thereof.

30. The composition of claim 22, wherein the composition further comprises one or more additional pharmaceutically active compounds.

31. The composition of claim 22, wherein enriched (S)-bisoprolol is provided in a controlled-release formulation.

32. The composition according to claim 31, wherein the controlled-release formulation exhibits an in vitro dissolution profile substantially corresponding to the following, when measured by U.S. Pharmacopoeia (USP) Type 1 Apparatus (baskets) at 37° C. and 50 rpm in 0.01 N HCl for the first 2 hours, followed by transfer to phosphate buffer at pH 6.8 or higher for the remainder of the measuring period:
(a) from about 0% to about 10% of the total (S)-bisoprolol is released after 2 hours;
(b) less than about 50% of the total (S)-bisoprolol is released after 4 hours; and
(c) greater than about 50% of the total (S)-bisoprolol is released after 22 hours of measurement in said apparatus.

33. The formulation according to claim 31, wherein the controlled-release formulation exhibits an in vitro dissolution profile substantially corresponding to the following, when measured by USP Type 1 Apparatus (baskets) at 37° C. and 100 rpm in 0.01 N HCl for the first 2 hours, followed by transfer to phosphate buffer at pH 7.2 for the remainder of the measuring period:

| | |
|---|---|
| 0-2 hours | <10% released |
| 4 hours | <30% released |
| 6 hours | 10-40% released |
| 8 hours | 30-70% released |
| 12 hours | >80% released. |

34. The composition according to claim 31, which, upon administration to a human, exhibits (i) a first phase, during which plasma concentration of the (S)-bisoprolol is maintained at a sub-therapeutic level in the human for at least about 2 hours to about 10 hours following administration; followed by (ii) a second phase, during which the (S)-bisoprolol is released from the formulation such that the plasma concentration of the (S)-bisoprolol in the blood stream of the subject is maintained above a minimum therapeutic level for the remainder of a 24-hour period measured from administration.

35. The composition according to claim 34, wherein during the first phase the plasma concentration of (S)-bisoprolol ranges from about 0 to about 5 ng/ml.

36. The composition according to claim 34, wherein during the first phase the plasma concentration of (S)-bisoprolol is about 1 ng/ml.

37. The composition according to claim 35, wherein during the second phase the plasma concentration of (S)-bisoprolol ranges from about 2 to about 50 ng/ml.

38. A method of treating one or more cardiovascular conditions comprising administering a therapeutically effective amount of enriched (S)-bisoprolol, or a pharmaceutically acceptable salt thereof, to a subject in need of such a treatment, wherein the subject obtains a therapeutic benefit resulting from the administration of enriched (S)-bisoprolol, and wherein the amount of enriched (S)-bisoprolol, or pharmaceutically acceptable salt thereof, is less than the amount of racemic bisoprolol required to achieve the same therapeutic benefit.

39. A method of reducing one or more side effects associated with racemic bisoprolol comprising administering a therapeutically effective amount of enriched (S)-bisoprolol, or a pharmaceutically acceptable salt thereof, to a subject in need of such a reduction, wherein one or more side-effects are reduced relative to those resulting from the administration of an equivalent amount of racemic bisoprolol.

40. A method of reducing one or more drug interactions associated with administration of racemic bisoprolol comprising administering a therapeutically effective amount of enriched (S)-bisoprolol, or a pharmaceutically acceptable salt thereof, to a subject in need of such a reduction, wherein one or more drug interactions are reduced relative to those resulting from the administration of an equivalent amount of racemic bisoprolol.

41. A method of reducing oxidative metabolism by the cytochrome P450 isoform CYP2D6 associated with racemic bisoprolol comprising administering a therapeutically effective amount of enriched (S)-bisoprolol, or a pharmaceutically acceptable salt thereof, to a subject in need of such a reduction, wherein the amount of oxidative metabolism by the cytochrome P450 isoform CYP2D6 in the subject is reduced relative to the amount of oxidative metabolism by the cytochrome P450 isoform CYP2D6 in a subject receiving an equivalent amount of racemic bisoprolol.

42. A method of preventing one or more cardiovascular conditions treatable with racemic bisoprolol, comprising administering a therapeutically effective amount of enriched (S)-bisoprolol, or a pharmaceutically acceptable salt thereof, to a subject in need of such prevention, wherein the administration of enriched (S)-bisoprolol, or pharmaceutically acceptable salt thereof, reduces one or more side-effects relative to a racemic mixture of bisoprolol.

43. A method of managing one or more cardiovascular conditions comprising administering a therapeutically effective amount of enriched (S)-bisoprolol, or a pharmaceutically acceptable salt thereof, to a subject in need of such management, wherein the administration of enriched (S)-bisoprolol, or pharmaceutically acceptable salt thereof, reduces one or more side-effects relative to a racemic mixture of bisoprolol.

44. A method of extending the therapeutic effect of a treatment for one or more cardiovascular conditions comprising administering a therapeutically effective amount of enriched (S)-bisoprolol, or a pharmaceutically acceptable salt thereof, to a subject in need of such treatment, wherein the administration of enriched (S)-bisoprolol, or pharmaceutically acceptable salt thereof, provides a therapeutic effect that lasts longer than the therapeutic effect achieved by administration of an equal amount of racemic bisoprolol.

45. A method of reducing the variability in metabolism of racemic bisoprolol associated with polymorphic expression of cytochrome P450 isoform CYP2D6, comprising administering a therapeutically effective amount of enriched (S)-bisoprolol, or a pharmaceutically acceptable salt thereof, to a subject in need of such a reduction, wherein the variability in metabolism by cytochrome P450 isoform CYP2D6 in the subject is reduced relative to the variability in metabolism by the cytochrome P450 isoform CYP2D6 in a subject receiving an equivalent amount of racemic bisoprolol.

46. A method of reducing the effects of inducers or inhibitors of cytochrome P450 isoform CYP2D6 comprising administering a therapeutically effective amount of enriched (S)-bisoprolol, or a pharmaceutically acceptable salt thereof, to a subject in need of such a reduction, wherein the effects of inducers or inhibitors of cytochrome P450 isoform CYP2D6 in the subject is reduced relative to the effects of inducers or inhibitors of cytochrome P450 isoform CYP2D6 in a subject receiving an equivalent amount of racemic bisoprolol.
